(19) United States
(12) Patent Application Publication
(10) Pub. No.: US 2023/0052984 A1
(43) Pub. Date: Feb. 16, 2023

(54) SYSTEM AND METHOD FOR EXERCISE ASSESSMENT AND PRESCRIPTION GENERATION

(71) Applicant: PREVENTIVE MEDICAL HEALTH CARE CO., LTD., Taichung City (TW)

(72) Inventor: Ching-Yu YEH, TAICHUNG (TW)

(21) Appl. No.: 17/818,710

(22) Filed: Aug. 9, 2022

(30) Foreign Application Priority Data
Aug. 10, 2021 (TW) 110129499

(51) Int. Cl. A63B 24/00; G16H 20/30

(52) U.S. Cl. CPC A63B 24/0075; G16H 20/30

(57) ABSTRACT

A system for exercise assessment and prescription generation, which is signally connected with at least one exercise device includes a biological information collecting module, an assessing standard generating module, an exercising information collecting module and a personal prescription generating module. The biological information collecting module collects biological information of a user. The assessing standard generating module is signally connected with the biological information collecting module and a cloud data platform, and for generating an assessing standard by calculating the biological information and the plurality of reference information in the cloud data platform. The exercising information collecting module is signally connected with at least one exercise device, and obtains assessed exercising information.

### RELATED APPLICATIONS

This application claims priority to Taiwan Application Serial Number 110129499, filed Aug. 10, 2021, which is herein incorporated by reference.

### BACKGROUND

### Technical Field

The present disclosure relates to a system and method for information processing. In particular, the present disclosure relates to a system and method for exercise assessment and prescription generation.

### Description of Related Art

In recent years, as health awareness of the public has increased and more and more people are concerned about the issue of figure and body sculpting, fitness has become a basic way of exercise. On the contrary, the fitness group gets more serious about the result and how to achieve their goal effectively by exercise.

In the general fitness center, the coach will make an exercise prescription according to the physical condition and performance of the trainee when the trainee wants to achieve a specific goal. The trainee can execute the exercising prescription with the exercise device to achieve their goal effectively and avoid sports injury. However, the coach can only provide exercise prescriptions according to personal performance observations of the trainee, and they often elevate the quantity of motion in order to make them aware of the effect of exercise. Therefore, what the trainee actually needs to know is what exercise prescription is suitable for them, so that they can achieve their exercise goal effectively. Further, trainee may suffer sports injury easily when a coach is not present during exercise.

### SUMMARY

According to one aspect of the present disclosure, a system for exercise assessment and prescription generation, which is signally connected with at least one exercise device, which includes a biological information collecting module, an assessing standard generating module, an exercising information collecting module, and a personal prescription generating module. The biological information collecting module collects the biological information of a user. The assessing standard generating module is signally connected with the biological information collecting module and a cloud data platform, and generates an assessing standard by calculating the biological information and a plurality of reference information in the cloud data platform. The exercising information collecting module is signally connected with at least one exercise device and obtains assessed exercising information. The personal prescription generating module is signally connected with the assessing standard generating module and the exercising information collecting module, which generates a personal exercise prescription by comparing the assessed exercising information with the assessing standard and, wherein at least one exercise device is set up according to the personal exercise prescription and the user uses the at least one exercise device according to the personal exercise prescription. The assessed exercising information includes an exercise range, a resistance value, an exercise time, a total number of times, a number of effective times and a speed value.

According to another aspect of the present disclosure, a method for exercise assessment and prescription generation includes acquiring biological information of a user, calculating the biological information with a plurality of reference information in a cloud data platform and generating an assessing standard, obtaining assessed exercising information and comparing the assessed exercising information with the assessing standard and generating a personal exercise prescription. At least one exercise device is set according to the personal exercise prescription and the user uses at least one exercise device according to the personal exercise prescription. The assessed exercising information includes an exercise range, a resistance value, an exercise time, a total number of times, a number of effective times and a speed value.

### BRIEF DESCRIPTION OF THE DRAWINGS

The present disclosure can be better understood by reading the following detailed description of the embodiment, with reference made to the accompanying drawings as follows:

FIG. 1 is a flow chart of a method for exercise assessment and prescription generation according to an embodiment of the present disclosure.

FIG. 2 is a block diagram of a system for exercise assessment and prescription generation according to another embodiment of the present disclosure.

FIG. 3 is a schematic view of a display screen of the exercise device according to the embodiment of FIG. 2.

FIG. 4 is a schematic view of a user interface of the system for exercise assessment and prescription generation according to the embodiment of FIG. 2.

FIG. 5 is a schematic view of an exercise device that collocates with the system for exercise assessment and prescription generation according to the embodiment of FIG. 2.

FIG. 6 is a schematic view of another exercise device that collocates with the system for exercise assessment and prescription generation according to the embodiment of FIG. 2.

FIG. 7 is a schematic view of another exercise device that collocates with the system for exercise assessment and prescription generation according to the embodiment of FIG. 2.

FIG. 8 is a schematic view of another exercise device which collocates with the system for exercise assessment and prescription generation according to the embodiment of FIG. 2.

FIG. 9 is a schematic view of another exercise device which collocates with the system for exercise assessment and prescription generation according to the embodiment of FIG. 2.

FIG. 10 is a schematic view of another exercise device which collocates with the system for exercise assessment and prescription generation according to the embodiment of FIG. 2.

FIG. 11 is a schematic view of another exercise device which collocates with the system for exercise assessment and prescription generation according to the embodiment of FIG. 2.

FIG. 12 is a schematic view of another exercise device which collocates with the system for exercise assessment and prescription generation according to the embodiment of FIG. 2.

FIG. 13 is a schematic view of the user interface of the system for exercise assessment and prescription generation according to another embodiment of the present disclosure.

FIG. 14 is another schematic view of a user interface of the system for exercise assessment and prescription generation according to another embodiment of the present disclosure.

FIG. 15 is a schematic view of a user interface of the instant adjustment exercising prescription module according to the embodiment of FIG. 13.

FIG. 16 is a schematic view of the user interface of the instant adjustment exercising prescription module after operated according to the embodiment of FIG. 15.

### DETAILED DESCRIPTION

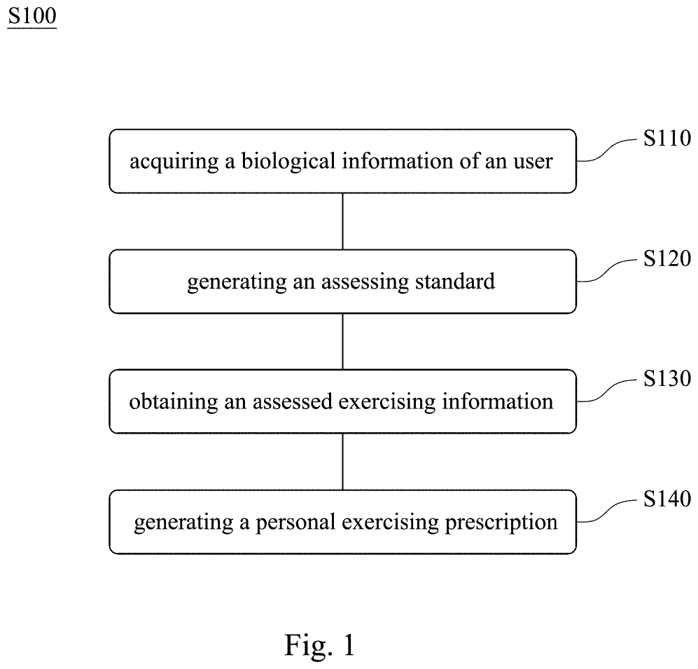

FIG. 1 is a flow chart of a method for exercise assessment and prescription generation S100 according to an embodiment of the present disclosure. In FIG. 1, the method for exercise assessment and prescription generation S100 is corresponded with at least one exercise device and includes the following steps. In Step S110, a user's biological information is acquired. In Step S120, the biological information is calculated with a plurality of reference information in a cloud data platform and an assessing standard is generated. In detail, the reference information in the cloud data platform is acquired by collecting the biological information extensively, and uses the ways of algorithm analysis, such as big data, AI, etc., to calculate, induct, and generate the assessing standard, which is favorable for quantizing the exercise performance, finding out the rhythmicity, and then grading the exercise performance in different level to make the assessing standard. In Step S130, an assessed exercising information is acquired. In detail, the user who has a demand of exercising prescription or exercise recommendation can operate the exercise device and generate the assessed exercising information, and for applying to different types of exercise device, the assessed exercising information includes an exercise range, a resistance value, an exercise time, a total number of times, a number of effective times and a speed value. Further, when the user uses other exercise device, the assessed exercising information can include other judgement data, but is not limited thereto. In Step S140, the assessed exercising information is compared with the assessing standard and a personal exercise prescription is generated. The personal exercise prescription can provide a suggestion for the user about how to use the exercise device properly, such as operating time, operating speed, etc., and how to set the parameter of the exercise device properly, such as the resistance, etc. Therefore, it is favorable for offering the user an exercise recommendation more objectively by quantifying the exercise performance and substituting the human exercising prescription by the AI exercising prescription to improve user's body movement in different ways, such as activity, coordination, exercise accuracy, etc.

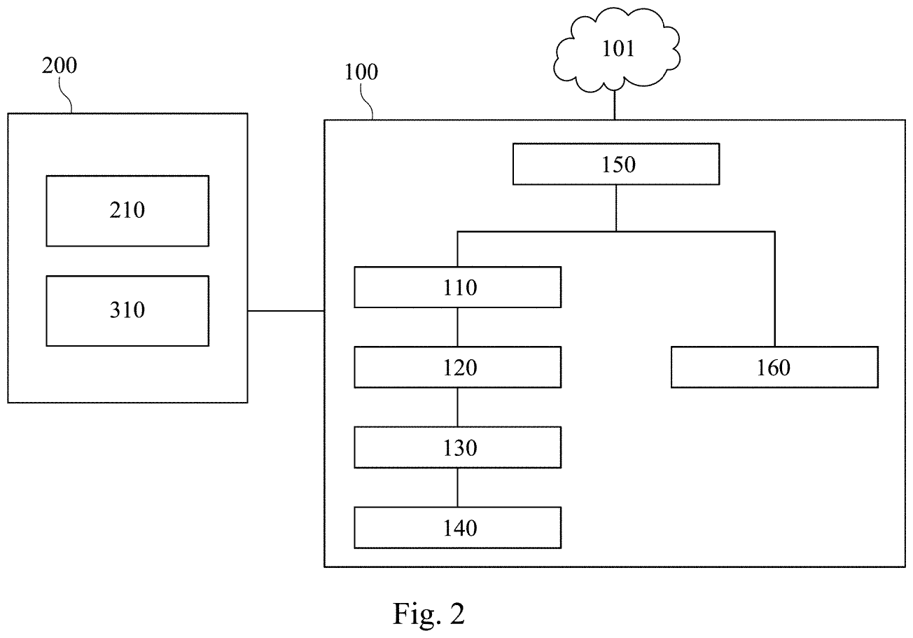

FIG. 2 is a block diagram of a system for exercise assessment and prescription generation 100 according to another embodiment of the present disclosure. The method for exercise assessment and prescription generation S100 in FIG. 1 can correspond with the system for exercise assessment and prescription generation 100, but is not limited thereto.

In FIG. 2, the system for exercise assessment and prescription generation 100 is signally connected with an exercise device 200 and includes a biological information collecting module 110, an assessing standard generating module 120, an exercising information collecting module 130 and a personal prescription generating module 140. The biological information collecting module 110 collects the biological information of a user. The assessing standard generating module 120 is signally connected with the biological information collecting module 110 and a cloud data platform 101, and generates an assessing standard by calculating the biological information and a plurality of reference information in the cloud data platform 101. The exercising information collecting module 130 is signally connected with the exercise device 200, and obtains assessed exercising information. The personal prescription generating module 140 is signally connected with the assessing standard generating module 120 and the exercising information collecting module 130, which generates a personal exercise prescription by comparing the assessed exercising information with the assessing standard. The exercise device 200 is set up according to the personal exercise prescription and the user operates the exercise device 200 according to the personal exercise prescription. The assessed exercising information includes exercise range, resistance value, exercise time, total number of times, number of effective times, and speed value. The personal exercise prescription includes exercise time recommendation information, exercise device resistance value setting information, exercise device swing range setting information, and exercise speed setting information, but is not limited thereto. The personal exercise prescription can be adjusted with different exercise devices. The personal exercise prescription can be displayed on the display screen 210 of the exercise device 200, so that the user can operate the exercise device 200 according to the personal exercise prescription.

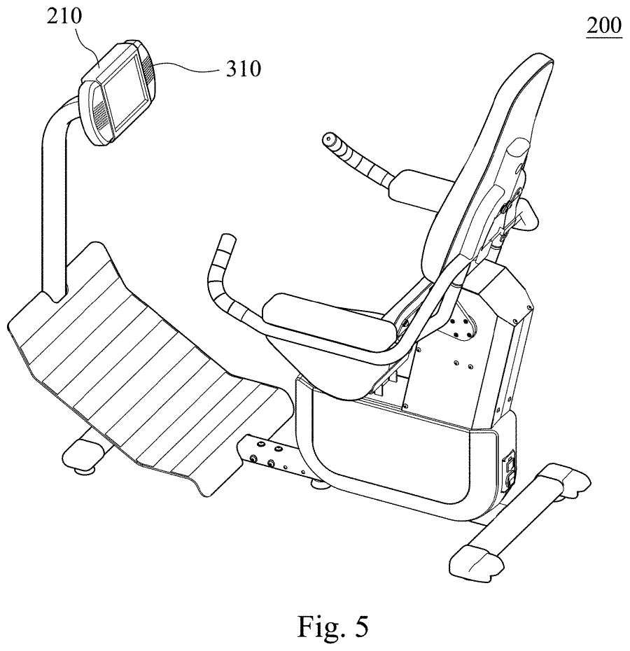

For example, when the exercise device 200 is a core exercise device (as shown in FIG. 5), the assessed exercising information can include the information in Table 1.

TABLE 1

| | |
|---|---|
| Date | 2019 May 14 |
| Exercise time | 02 min 00 sec |
| Correct times | 61 times |
| Average movement angle | 27 degrees |
| Average energy consumption | 10 Watt |
| Cumulative energy consumption | 0.7 Kilowatt |
| Correct repeat rate | 87.1% |
| Coordination | 60% |
| Average error angle | 1 degree |

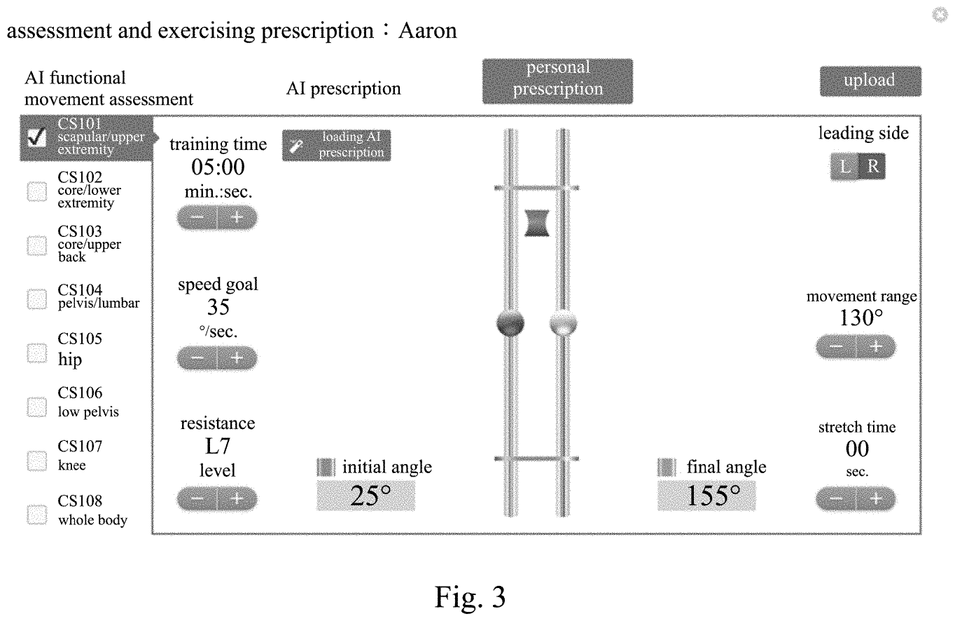

Since the exercising information can be recorded by the exercising information collecting module 130 and stored in the cloud data platform 101 after exercise, the exercising information can be the reference information of the cloud data platform 101. After collecting an abundance of exercising information, it becomes a favorable standard for assessing exercise performance according to the coordination (fluency), correct repeats rate, accuracy (error angle) and operated times. For example, the highest level is grade A, which represents coordination at 95%-100%, correct repeat rate at 90%-100%, error angle at 1 degree-2 degrees and operating times at 91 times-100 times. FIG. 3 is a schematic view of a display screen 210 of the exercise device 200 according to the embodiment of FIG. 2. When the assessed exercising information is assessed as grade A, it indicates that the user performs well on coordination, exercise accuracy, and activity, which suggests the module to offer a more difficult exercise. The personal exercise prescription can offer the recommendation of the setting the training time at 5 min/times, speed goal at 35 degrees/sec, exercise range at 130 degrees (the initial angle is 25 degrees and the final angle is 155 degrees) according to assessing standard and assessed exercising information, and set the resistance to a higher level (such as level L7). The system and method for exercise assessment and prescription generation of the present disclosure not only elevate the quantity of motion of user, but also further improve the exercise quality adaptively by the personal exercise prescription.

Further, the aforementioned exercise range in the assessed exercising information is related with the average exercise angle. The resistance value (level of resistance) is provided by each exercise device 200. The exercise time is corresponded to the exercise time in Table 1. The number of effective times is the correct times in Table 1. The correct repeats rate is acquired by calculating the rate of correct times in the total number of times. The speed value can acquire by calculating the average movement angle and the exercise time in Table 1.

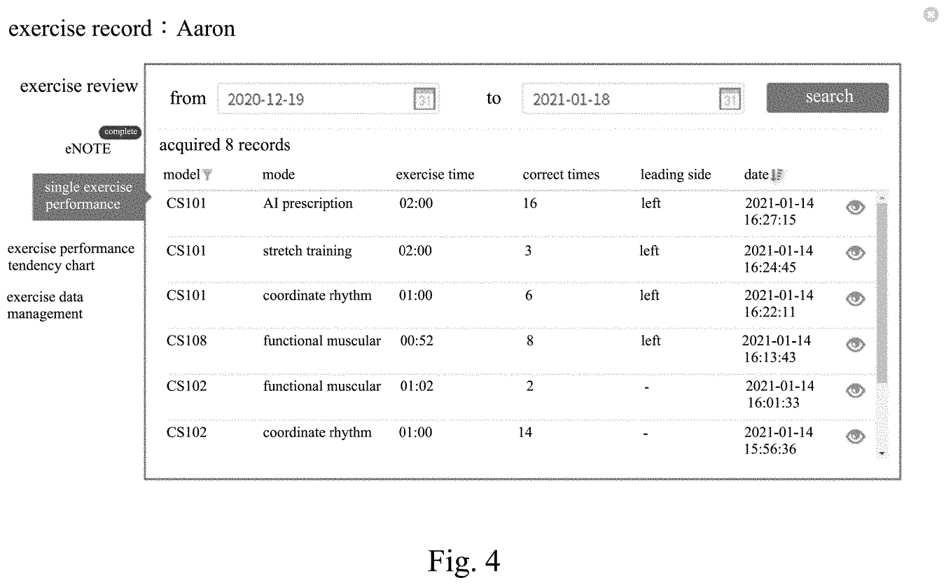

FIG. 4 is a schematic view of a user interface of the system for exercise assessment and prescription generation 100 according to the embodiment of FIG. 2. In FIG. 2 and FIG. 4, the system for exercise assessment and prescription generation 100 can further include a functional prescription generating module 160. The functional prescription generating module 160 generates at least one functional exercising prescription according to the reference information in the cloud data platform 101, which is favorable for offering the user a way to use the exercise device 200 according to the functional exercising prescription. That is, the system for exercise assessment and prescription generation 100 is not only for offering a customized personal exercise prescription, but also offer or offering a functional exercising prescription according to the assessing standard. Hence, a user who doesn't have a long-term exercise plan can exercise on demand by choosing corresponding functional exercising prescription, such as quick exercise, physical fitness challenge, stretching, functional muscular strength training, coordination, etc. Therefore, a user can follow the suggestion and achieve the exercise demand effectively and instantly.

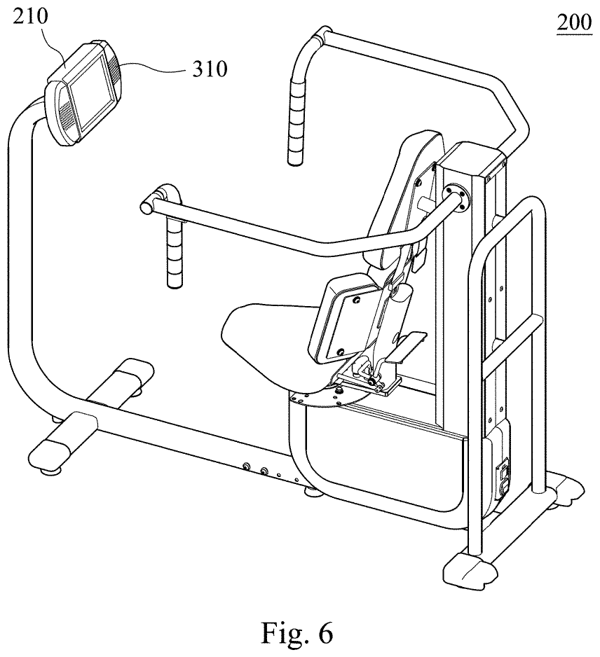

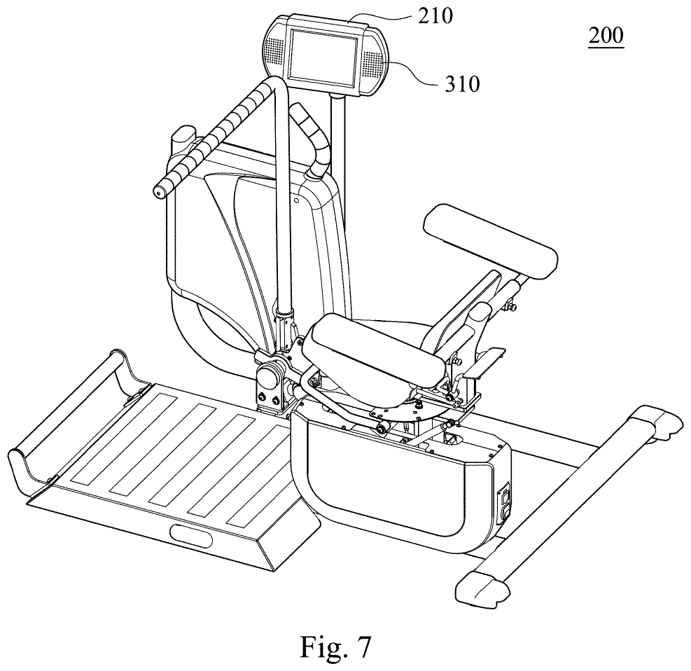

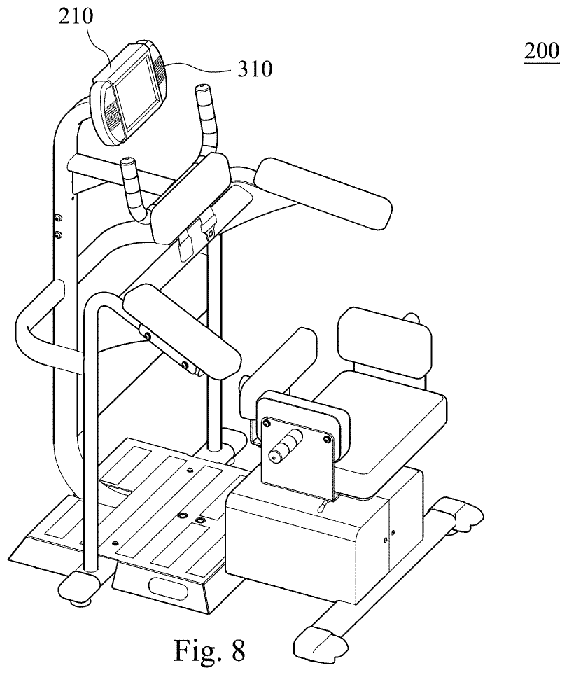

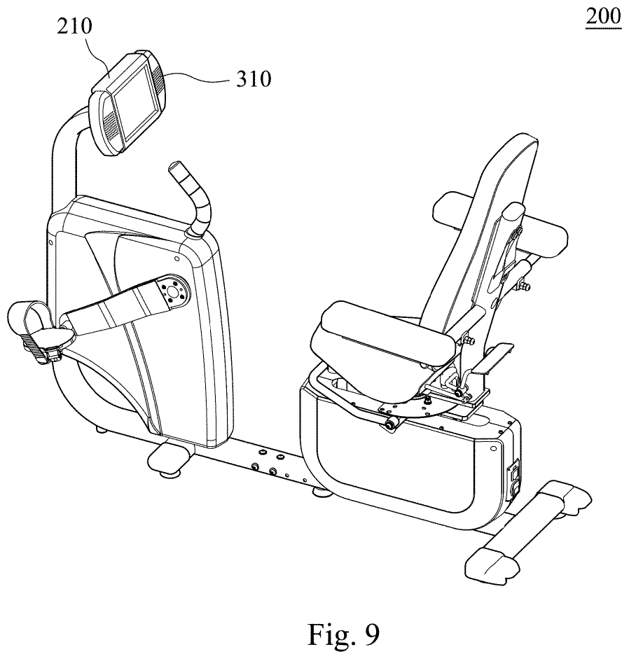

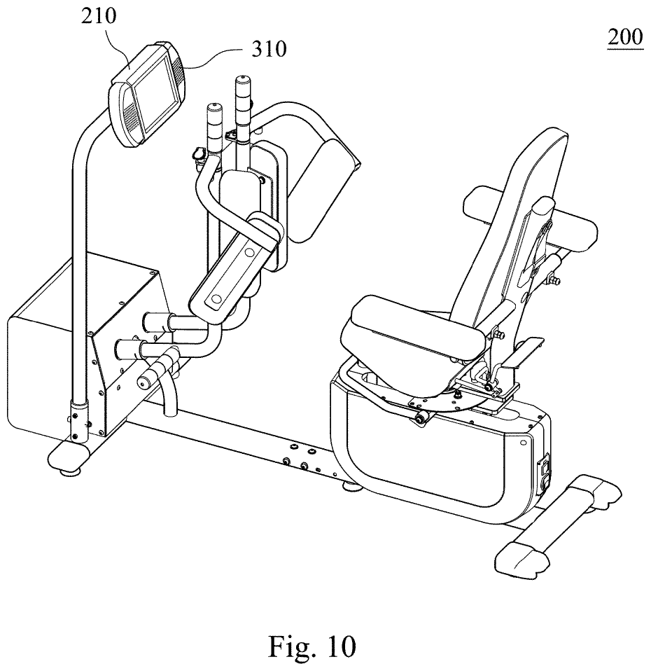

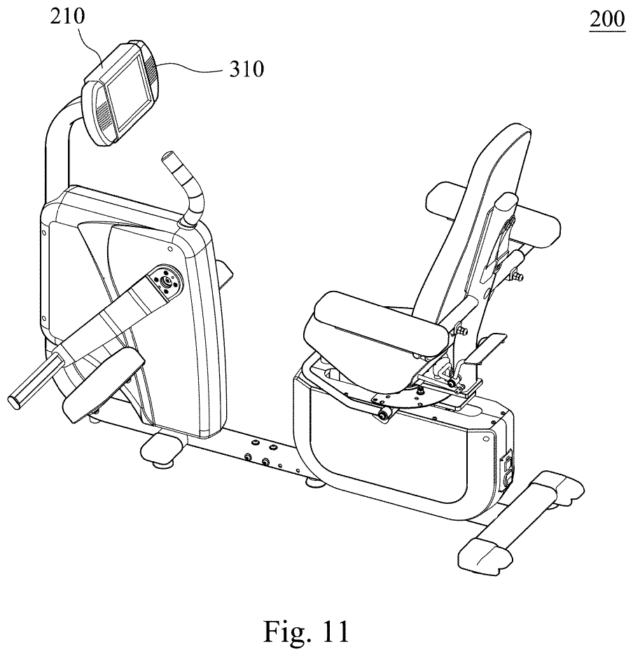

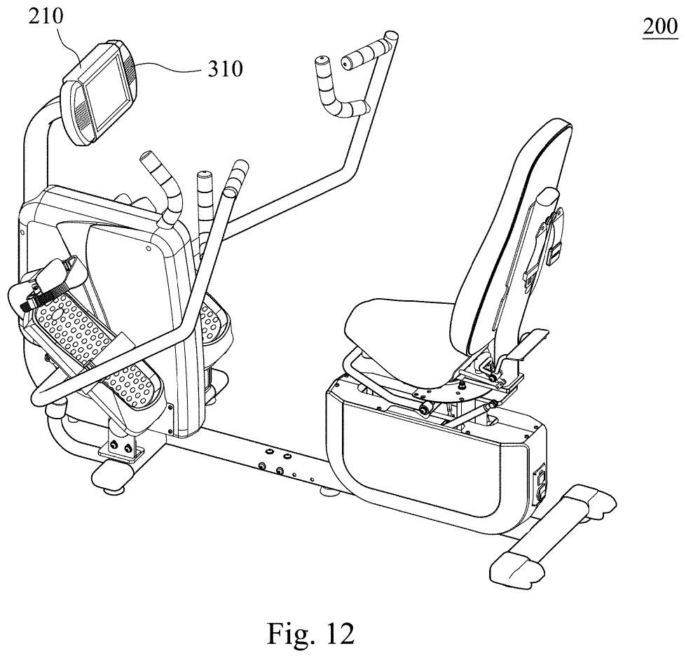

Please refer to FIG. 5 to FIG. 12. FIG. 5 is a schematic view of an exercise device 200 which collocates with the system for exercise assessment and prescription generation 100 according to the embodiment of FIG. 2. FIG. 6 is a schematic view of another exercise device 200 which collocates with the system for exercise assessment and prescription generation 100 according to the embodiment of FIG. 2. FIG. 7 is a schematic view of another exercise device 200 which collocates with the system for exercise assessment and prescription generation 100 according to the embodiment of FIG. 2. FIG. 8 is a schematic view of another exercise device 200 which collocates with the system for exercise assessment and prescription generation 100 according to the embodiment of FIG. 2. FIG. 9 is a schematic view of another exercise device 200 which collocates with the system for exercise assessment and prescription generation 100 according to the embodiment of FIG. 2. FIG. 10 is a schematic view of another exercise device 200 which collocates with the system for exercise assessment and prescription generation 100 according to the embodiment of FIG. 2. FIG. 11 is a schematic view of another exercise device 200 which collocates with the system for exercise assessment and prescription generation 100 according to the embodiment of FIG. 2. FIG. 12 is a schematic view of another exercise device 200 which collocates with the system for exercise assessment and prescription generation 100 according to the embodiment of FIG. 2. In order to provide the user with better exercise quality through personal exercise prescription, a number of the exercise device 200 which is favorable for corresponding with the system and method for exercise assessment and prescription generation is eight. In FIG. 5 to FIG. 12, the exercise device 200 includes a display screen 210, an interface (as shown in FIG. 3) of the system for exercise assessment and prescription generation 100 can display on the display screen 210. Therefore, it is favorable for the user to exercise according to the personal exercise prescription by operating the exercise device 200. In detail, the exercise device 200 in FIG. 5 is a core exercise device (the interface corresponded to FIG. 3 is a core strengthening machine). The exercise device 200 in FIG. 6 is a scapular exercise device (the interface corresponded to FIG. 3 is a scapular activation machine). The exercise device 200 in FIG. 7 is a balance exercise device (the interface corresponded to FIG. 3 is a balance strengthening machine). The exercise device 200 in FIG. 8 is a pelvis exercise device (the interface corresponded to FIG. 3 is a pelvis strengthening machine). The exercise device 200 in FIG. 9 is a hip exercise device (the interface corresponded to FIG. 3 is a hip strengthening machine). The exercise device 200 in FIG. 10 is a low pelvis exercise device (the interface corresponded to FIG. 3 is a low pelvis strengthening machine). The exercise device 200 in FIG. 11 is a knee exercise device (the interface corresponded to FIG. 3 is a knee strengthening machine). The exercise device 200 in FIG. 12 is a whole body exercise device (the interface corresponded to FIG. 3 is a whole body strengthening machine), but is not limited thereto. In other words, the exercise devices include the training from shoulder to feet, which can collocate with the system and method for exercise assessment and prescription generation of the present disclosure, it is favorable for providing the personal prescription generating module with different exercise devices adaptively according to the user's physical activity condition. For example, the personal prescription generating module 140 can assess grade C according to the assessed exercising information from the scapular exercise device and assess level A according to the assessed exercising information from the hip exercise device for the same person. That is, the personal prescription generating module 140 is favorable for providing different exercise recommendation to the different items according to the personal exercise prescription.

It is worth to mention that the generation of the assessing standard that corresponds to the different exercise devices is based on coordination, correct repeats rate, accuracy (error angle), and operating times, which then adjusts the ratio to assess according to the characteristic of the exercise device. For example, the whole body exercise device focuses on the coordination of the user, so that the ratio of the coordination is 40 percent and the accuracy is 10 percent. The scapular exercise device focuses on stretching the scapular muscle group through the accuracy of motion and prevents the sports injury, so that the ratio of the coordination is 35 percent, which is the highest ratio. The system for exercise assessment and prescription generation 100 can further include a user management module 150 for storing a user's information, wherein the biological information, the assessed exercising information, and the personal exercise prescription are stored in the cloud data platform 101 in correspondence to the user's information. The system for exercise assessment and prescription generation 100 can process and store an amount of the reference information by connecting with the cloud data platform 101, which is favorable for providing for the exercise center, the fitness center or the physical therapy and rehabilitation clinic, and the hospital to use. Therefore, it is favorable for elevating the customize functionality and convenience of the system for exercise assessment and prescription generation 100 by storing and setting up the user information through the user management module 150. Further, the system for exercise assessment and prescription generation 100 is favorable for confirming the identification of the user and providing different access rights according to the different user information. For example, the normal user, such as the body shaper and the physical therapy patient only have the right to enter the system for exercise assessment and prescription generation 100 to confirm their user information, record and personal exercise prescription, etc. But the coach or the physical therapy doctor has the right to see the user information of client, patient or themselves.

In order to elevate the convenience of the system for exercise assessment and prescription generation 100, the system for exercise assessment and prescription generation 100 can signally connect with a radio frequency identification device. The radio frequency identification device includes a reading device 310 and a radio frequency identification element (the number is omitted). Please refer to FIG. 5 to FIG. 12, the reading device 310 can be disposed on the side of the display screen 210, so that it is favorable for the user to operate. The reading device 310 is electrically connected with the exercise device 200 or the computer which is for the system for exercise assessment and prescription generation 100 to apply thereon. The radio frequency identification element is for the reading device 310 to induce, and loads the corresponded user information into the exercise device 200 or the computer. When the user exercises according to the personal exercise prescription, just needs to sense the reading device 310 of the exercise device 200 with the radio frequency identification element and start to work out. Further, if the user wants to confirm their exercise record or generate a personal exercise prescription by using the biological information as the assessed exercising information, they can enter the system for exercise assessment and prescription generation 100 by connecting the radio frequency identification element on the reading device of the computer (the number is omitted).

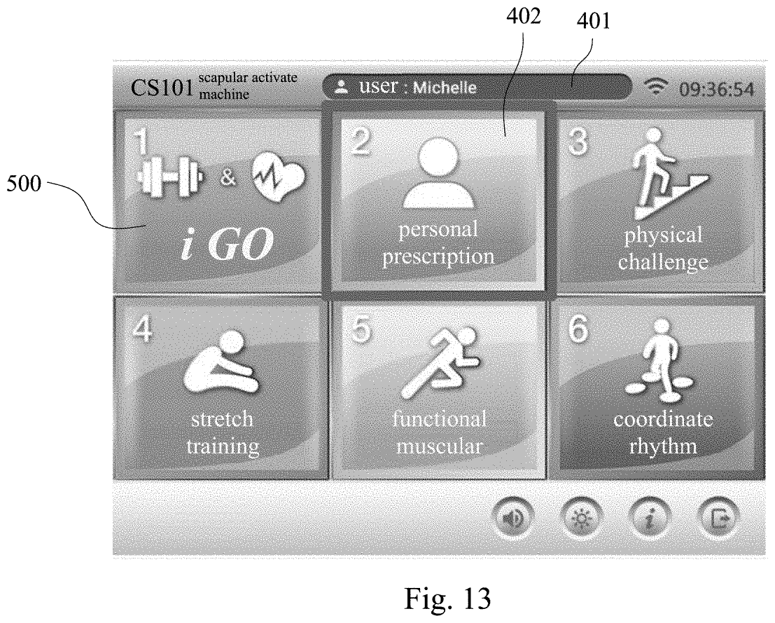

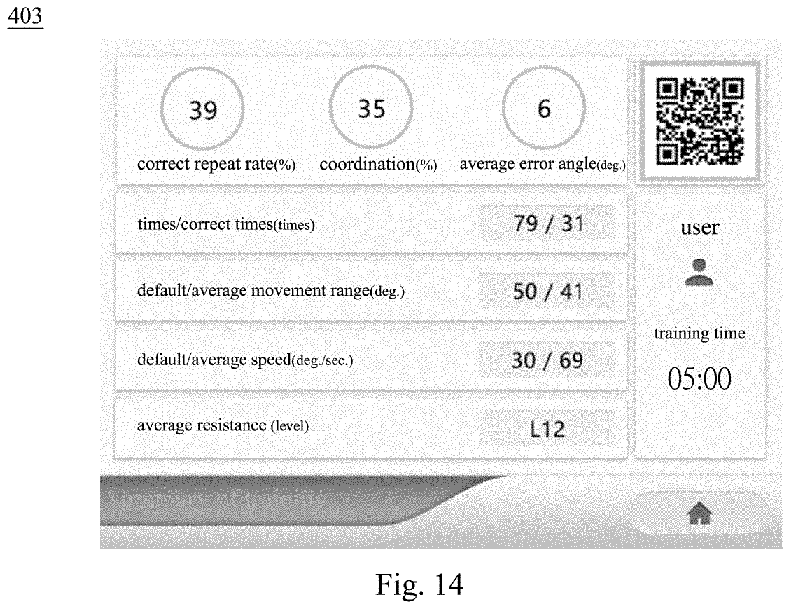

In detail, the radio frequency identification element has recorded a card number, which is favorable for reading the number and loading the user information which corresponded with the card number by the reading device 310. FIG. 13 is a schematic view of the user interface of the system for exercise assessment and prescription generation according to another embodiment of the present disclosure. FIG. 14 is another schematic view of a user interface of the system for exercise assessment and prescription generation according to another embodiment of the present disclosure. In FIG. 13, it is favorable for loading the user information of a user 401 by the radio frequency identification element sensing the reading device 310. The user information can be loaded to correspond to the personal exercise prescription 402, therefore, it is favorable for providing the user a recommendation of exercise according to the personal exercise prescription 402 so as to obtain a new comparison evaluation result 403, but is not limited thereto.

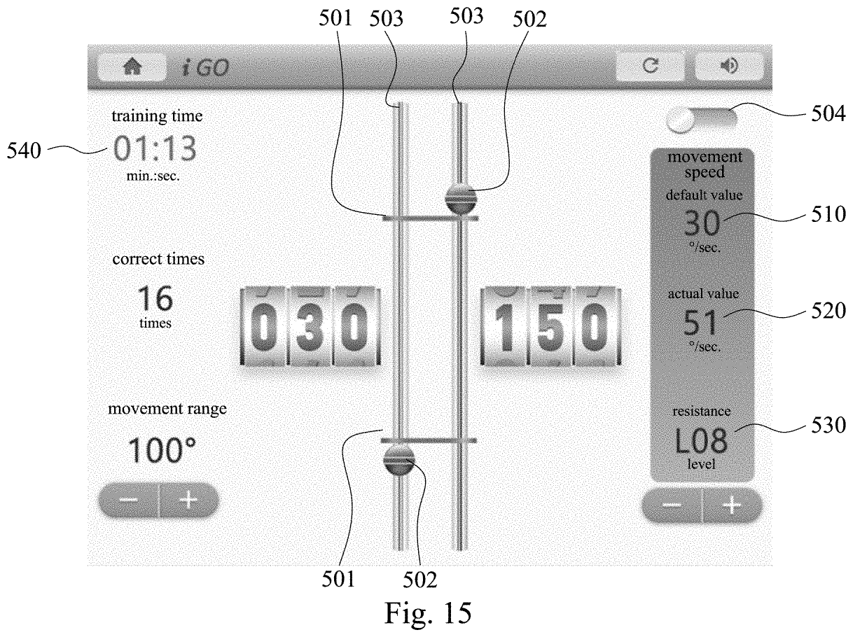

Please refer to FIG. 13, the system for exercise assessment and prescription generation can further provide an instant adjustment exercising prescription module 500. FIG. 15 is a schematic view of a user interface of the instant adjustment exercising prescription module 500 according to the embodiment of FIG. 13. An angled line 501 is a turning point of the correct times. When the user's left and right feet or left and right hand operates the exercise device back and forth, a ball 502 will move along a basic line 503, and the user should operate reversely when the ball 502 touches the angled line 501, which is a correct way of operating (a back and forth correctly can be count as a correct time). Further, the instant adjustment exercising prescription module 500 is favorable for providing a default value 510 so as to detect and display an actual value 520 in the process of exercise of the user. When the actual value 520 is greater than the default value 510, it is favorable for providing a command to the exercise device to promote the resistance value 530. When the actual value 520 is less than the default value 510, it is favorable for providing a command to the exercise device to lower the resistance value 530. Further, the function of adjusting the resistance value instantly can be turned on/off with the switch 504 on the upper right side of the interface.

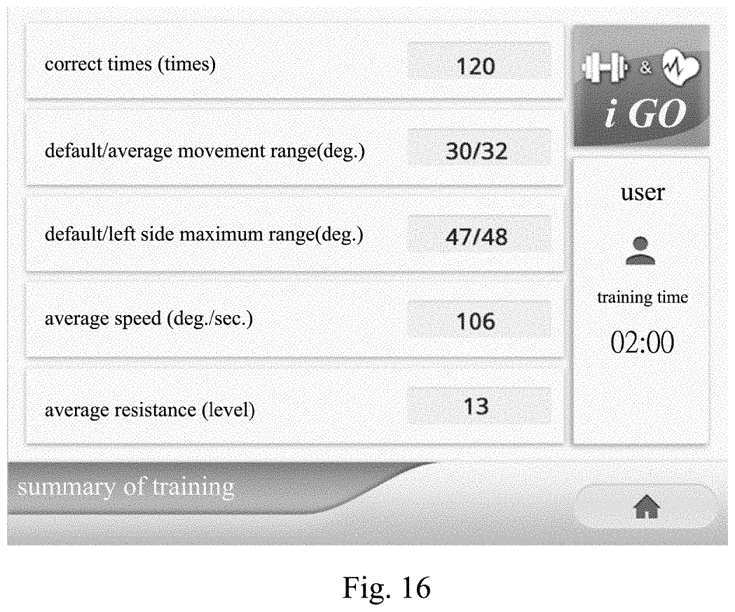

FIG. 16 is a schematic view of the user interface of the instant adjustment exercising prescription module 500 after operated according to the embodiment of FIG. 15. In FIGS. 15 and 16, after the user exercise according to the default training time 540 will generate an exercise result as shown in FIG. 16. The exercise result will show the correct times, the maximum angle on the left/right side, the average speed and average resistance in the process of exercise, and the exercise result will list the default movement range and the average movement range in the process of exercise. Therefore, it is favorable for the user to find out their performance of their functional muscular, muscular endurance and physical strength.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A system for exercise assessment and prescription generation, which is signally connected with at least one exercise device, comprising:
a biological information collecting module collecting biological information of a user;
an assessing standard generating module signally connected with the biological information collecting module and a cloud data platform, and for generating an assessing standard by calculating the biological information and a plurality of reference information in the cloud data platform;
an exercising information collecting module signally connected with the at least one exercise device, and obtaining assessed exercising information; and
a personal prescription generating module signally connected with the assessing standard generating module and the exercising information collecting module, which is for generating a personal exercise prescription by comparing the assessed exercising information with the assessing standard and, wherein the at least one exercise device is set up according to the personal exercise prescription and the user uses the at least one exercise device according to the personal exercise prescription;
wherein the assessed exercising information comprises an exercise range, a resistance value, an exercise time, a total number of times, a number of effective times, and a speed value.

2. The system for exercise assessment and prescription generation of claim 1, further comprising:
a user management module for storing at least one user information;
wherein the biological information, the assessed exercising information and the personal exercise prescription are stored in the cloud data platform corresponding to the at least one user information.

3. The system for exercise assessment and prescription generation of claim 2, wherein the system for exercise assessment and prescription generation is signally connected with a radio frequency identification device, the radio frequency identification device comprises:
a reading device electrically connected with the at least one exercise device or a computer which is for the system for exercise assessment and prescription generation to apply thereon; and
a radio frequency identification element for the reading device to induce, and load the user information into the at least one exercise device or the computer.

4. The system for exercise assessment and prescription generation of claim 1, wherein a number of the at least one exercise device is eight, which are a scapular exercise device, a core exercise device, a balance exercise device, a pelvis exercise device, a hip exercise device, a low pelvis exercise device, a knee exercise device, and a whole-body exercise device.

5. The system for exercise assessment and prescription generation of claim 1, further comprising:
a functional prescription generating module, generating a functional exercising prescription according to the reference information in the cloud data platform, wherein the user uses the at least one exercise device according to the functional exercising prescription.

6. The system for exercise assessment and prescription generation of claim 1, wherein the personal exercise prescription comprises an exercise time recommendation information, an exercise device resistance setting information, an exercise device swing range setting information, and an exercise speed setting information.

7. The system for exercise assessment and prescription generation of claim 1, wherein the personal exercise prescription displays on a display screen of the at least one exercise device.

8. A method for exercise assessment and prescription generation, comprising:
acquiring biological information of a user;
calculating the biological information with a plurality of reference information in a cloud data platform and generating an assessing standard;
obtaining assessed exercising information; and
comparing the assessed exercising information with the assessing standard and generating a personal exercise prescription;
wherein at least one exercise device is set according to the personal exercise prescription and the user uses the at least one exercise device according to the personal exercise prescription;
wherein the assessed exercising information comprises an exercise range, a resistance value, an exercise time, a total number of times, a number of effective times, and a speed value.

9. The method for exercise assessment and prescription generation of claim 8, wherein the method for exercise assessment and prescription generation generates at least one functional exercising prescription according to the reference information in the cloud data platform, and offers the user a way to use the at least one exercise device according to the at least one functional exercising prescription.
